(19) United States
(12) Patent Application Publication
(10) Pub. No.: US 2010/0233272 A1
(43) Pub. Date: Sep. 16, 2010

(54) DOSAGE FORMS COMPRISING CELECOXIB PROVIDING BOTH RAPID AND SUSTAINED PAIN RELIEF

(76) Inventors: Leah Elizabeth Appel, Bend, OR (US); Dwayne Thomas Friesen, Bend, OR (US); Scott M. Herbig, Groton, CT (US); Rodney James Ketner, Bend, OR (US); Sheri L. Shamblin, Groton, CT (US)

Correspondence Address: CHERNOFF, VILHAUER, MCCLUNG & STENZEL, LLP, 601 SW Second Avenue, Suite 1600, PORTLAND, OR, 97204-3157, US

(21) Appl. No.: 12/743,215

(22) PCT Filed: Nov. 6, 2008
(86) PCT No.: PCT/IB08/54640

§ 371 Date: May 14, 2010

Related U.S. Application Data
(60) Provisional application No. 60/988,179, filed on Nov. 15, 2007.

(51) Int. Cl. A61K 9/14; A61K 31/415

(52) U.S. Cl. 424/489; 514/406; 977/773; 977/906

(57) ABSTRACT

A pharmaceutical dosage form comprising celecoxib and a pharmaceutically acceptable carrier, the dosage form when initially administered to at least 12 human patients in the fasted state in a crossover study providing: (a) a mean blood plasma concentration of celecoxib within 0.5 hour after administration (C_{0.5}) of at least about 0.9 ng/ml per mg of celecoxib dosed; (b) a mean blood plasma concentration of celecoxib 12 hours after administration (Ci2) of at least about 0.6 ng/ml per mg of celecoxib dosed; (c) a mean area under the blood plasma concentration versus time curve for the 12 hour period following administration (AUC₁₂) of at least 19 ng-hr/mL per mg of celecoxib dosed; and (d) a mean maximum blood plasma concentration (Cₘₐₓ) of celecoxib of less than about 4.9 ng/ml per mg of celecoxib dosed.

### BACKGROUND OF THE INVENTION

The present invention relates to a dosage form comprising celecoxib that provides enhanced bioavailability, and both rapid and sustained pain relief.

Celecoxib is commercially available in capsule form with doses of 50 mg, 100 mg, 200 mg or 400 mg. While the commercial capsule provides efficacious blood levels of celecoxib to patients over a period of several hours, it has been observed in clinical studies that the amount of celecoxib absorbed by patients and which enters the blood stream is subject to a short time lag. Consequently, some patients do not experience pain relief until 30-60 minutes after the initial administration of the capsule. It has also been observed that there is some variability in the amount of celecoxib absorbed by patients during the initial one hour after administration, resulting in a fraction of patients not achieving efficacious blood levels of celecoxib until 60 minutes after administration of the dosage form.

It is therefore desired to provide a dosage form containing celecoxib that is capable of providing both immediate pain relief, as well as sustained pain relief. In addition, it is desired to reduce the amount of celecoxib in the dosage form relative to the commercial capsule, yet provide an equivalent exposure relative to the commercial capsule. It is also desired to provide immediate pain relief by providing initially high levels of celecoxib in the blood, but while also controlling the maximum concentration of celecoxib in the blood.

Desai et al., US Published Patent Application 2004/0242640 A1 disclose a dual release formulation of celecoxib comprising a fast release component and a slow release component. The fast release component is obtained by providing a first fraction of drug in the form of (1) solid particles having a D₅₀ less than 5 μm or (2) a solution in a pharmaceutically acceptable solvent. The second fraction of drug is in the form of (1) solid particles having a D₉₀ of greater than 5 μm or (2) any convenient particle size providing sustained release, slow release, programmed release, timed release, pulsed release, sustained release or extended release. However, the formulations exemplified by Desai et al., and shown in the plotted data, provide slower release in in vitro tests than that provided by the commercial capsule.

Accordingly, there is still a continuing need to formulate celecoxib to provide both rapid and sustained pain relief, particularly in treatment of acute disorders where early relief from pain or other symptoms is desired.

### BRIEF SUMMARY OF THE INVENTION

In a first aspect, a pharmaceutical dosage form comprises celecoxib and a pharmaceutically acceptable carrier. The dosage form when initially administered to at least 12 human patients in the fasted state in a crossover study provides:
(a) a mean blood plasma concentration of celecoxib within 0.5 hour after administration (C_{0.5}) of at least about 0.9 ng/ml per mg of celecoxib dosed;
(b) a mean blood plasma concentration of celecoxib 12 hours after administration (C₁₂) of at least about 0.6 ng/ml per mg of celecoxib dosed;
(c) a mean area under the blood plasma concentration versus time curve for the 12 hour period following administration (AUC₁₂) of at least 19 ng-hr/mL per mg of celecoxib dosed; and
(d) a mean maximum blood plasma concentration (Cₘₐₓ) of celecoxib of less than about 4.9 ng/ml per mg of celecoxib dosed.

In a second aspect, a pharmaceutical dosage form comprises celecoxib and a pharmaceutically acceptable carrier. The dosage form, when administered orally to at least 12 human patients in a crossover study, provides:
(a) a mean blood plasma concentration of celecoxib at 0.5 hour after administration (C_{0.5}) of at least 2.8 fold that provided by a control capsule;
(b) a mean blood plasma concentration of celecoxib 12 hours after administration (C₁₂) of at least 1.3 fold that provided by the control capsule;
(c) a mean area under the blood plasma concentration versus time curve for the 12 hour period following administration (AUC₁₂) of at least 1.7 fold that provided by the control capsule; and
(d) a mean maximum blood plasma concentration of celecoxib (Cₘₐₓ) of no greater than 2.6 fold that provided by the control capsule;
   wherein the control capsule consists of the same amount of celecoxib as the dosage form, but wherein the celecoxib is in crystalline form, and the control capsule further contains lactose, sodium lauryl sulfate, povidone, crosscarmellose sodium and magnesium stearate.

In a third aspect, a pharmaceutical dosage form comprises celecoxib and a pharmaceutically acceptable carrier, comprising (a) an immediate release portion and (b) a sustained release portion comprising celecoxib in a solubility-improved form.

In a fourth aspect, a pharmaceutical dosage form comprises celecoxib. The dosage form comprises (a) 25 wt % to 60 wt % of an immediate release portion comprising (i) 30 wt % to 80 wt % of a molecular dispersion of celecoxib and a polymer selected from the group consisting of hydroxypropyl methylcellulose and hydroxypropyl methylcellulose acetate succinate; (ii) 1 wt % to 15 wt % disintegrant; (iii) 20 wt % to 60 wt % diluent; and (iv) 0.05 wt % to 2 wt % lubricant; and (b) 40 wt % to 75 wt % of a sustained release portion comprising (i) 30 wt % to 80 wt % of a molecular dispersion of celecoxib and a polymer selected from the group consisting of hydroxypropyl methylcellulose and hydroxypropyl methylcellulose acetate succinate; (ii) 10 wt % to 50 wt % matrix material; (iii) 2 wt % to 40 wt % diluent; and (iv) 0.05 wt % to 2 wt % lubricant.

In one embodiment, the dosage form contains a solubility-improved form of celecoxib in an immediate release portion and a sustained release portion. Dosage forms containing solubility-improved forms of celecoxib in both an immediate and sustained release portion of the dosage form (1) provide a pharmacokinetic profile that provides both immediate and sustained pain relief; (2) reduce the amount of celecoxib needed to achieve pain relief while still providing sustained pain relief; (3) achieve about the same exposure as the commercial capsule but with a lower dose of celecoxib; and (4) maintain a Cₘₐₓ that is about the same as that achieved with the commercial capsule.

Thus, the dosage form provides several advantages over the prior art. The dosage form provides an immediate release of celecoxib so that efficacious blood levels of celecoxib are quickly achieved. Patients taking the dosage form of celecoxib obtain rapid pain relief due to the rapid initial release and absorption of celecoxib. Nevertheless, the dosage forms are also capable of providing sustained pain relief over a period of several hours.

Another advantage is a reduction in the variability of blood levels of celecoxib experienced by patients, particularly at the initial time periods after administration of the dosage form. Higher blood levels of celecoxib are more uniformly achieved, resulting in more patients achieving efficacious blood levels of celecoxib within the first hour after administration compared with the commercial capsule.

Yet another advantage is that a lower dose of celecoxib is utilized relative to the commercial capsule while still providing about the same exposure as the commercial capsule. Because celecoxib is in a solubility-improved form, the dosage forms improve the bioavailability of celecoxib, thereby allowing the total amount of celecoxib to be reduced. This in turn leads to another advantage, which is a reduction in patient to patient variability in blood levels of celecoxib achieved by the dosage forms. Since the variability in the amount of celecoxib absorbed by patients during the initial one hour after administration is reduced, more patients achieve efficacious blood levels of celecoxib within the first hour after administration of the dosage form than compared with the commercial capsule.

The foregoing and other objectives, features, and advantages of the invention will be more readily understood upon consideration of the following detailed description of the invention.

### BRIEF DESCRIPTION OF THE SEVERAL VIEWS OF THE DRAWINGS

FIG. 1 is a graph comparing the results of modeling work for two inventive dosage forms (DF-A and DF-B) and a control capsule.

FIG. 2. is a graph comparing the mean plasma celecoxib concentration versus time for a control capsule and the modeled mean plasma celecoxib concentration versus time.

### DETAILED DESCRIPTION OF THE INVENTION

In a first aspect, a pharmaceutical dosage form comprises celecoxib and a pharmaceutically acceptable carrier. The dosage form when initially administered to at least 12 human patients in the fasted state in a crossover study provides:
(a) a mean blood plasma concentration of celecoxib within 0.5 hour after administration (C_{0.5}) of at least about 0.9 ng/ml per mg of celecoxib dosed;
(b) a mean blood plasma concentration of celecoxib 12 hours after administration (C₁₂) of at least about 0.6 ng/ml per mg of celecoxib dosed;
(c) a mean area under the blood plasma concentration versus time curve for the 12 hour period following administration (AUC₁₂) of at least 19 ng-hr/mL per mg of celecoxib dosed; and
(d) a mean maximum blood plasma concentration of celecoxib (Cₘₐₓ) of less than 4.9 ng/ml per mg of celecoxib dosed.

By “initially administered” and “after initial administration” is meant after the initial or first dose of celecoxib is administered to a patient. Prior to the initial administration of celecoxib, the patient has not been administered celecoxib for a sufficient period of time so that the patient's blood plasma concentration of celecoxib is below the detectable limit. This is in contrast to measurement of celecoxib blood plasma concentrations in the steady state, in which the concentrations are measured after a sufficient number of administrations of the dosage form so that steady state blood plasma concentrations are achieved.

In order to provide rapid pain relief, the dosage form provides minimum blood levels of celecoxib within the first hour after the initial administration of the dosage form relative to the amount of celecoxib in the dosage form. In one embodiment, the dosage form provides a C_{0.5} of at least about 0.7 ng/ml per mg of celecoxib dosed. By “X ng/ml per mg of celecoxib dosed” is meant that for every mg of celecoxib in the dosage form, the dosage form will provide X ng/ml of celecoxib in the relevant fluid. For example, if the dosage form contains 140 mg of celecoxib, then a dosage form that provides a C_{0.5} of at least “about 0.7 ng/ml per mg of celecoxib dosed” will provide about a C_{0.5} of at least about 98 ng/ml. Preferably, the dosage form provides a C_{0.5} of at least about 0.75 ng/ml per mg of celecoxib dosed, more preferably at least about 0.8 ng/ml per mg of celecoxib dosed, and even more preferably at least about 0.9 ng/ml per mg of celecoxib dosed.

In another embodiment, the dosage form provides a mean blood plasma concentration of celecoxib within 1 hour after the initial administration (C₁) of at least about 1.6 ng/ml per mg of celecoxib dosed. More preferably, the dosage form provides a C₁ of at least about 1.8 ng/ml per mg of celecoxib dosed, and even more preferably of at least about 2 ng/ml per mg of celecoxib dosed.

In another embodiment, the dosage form provides a time to achieve the maximum concentration of drug in the blood plasma (Tₘₐₓ) of less than 5 hours following initial administration. Preferably, the dosage form provides a Tₘₐₓ of less than 4 hours.

In another embodiment, the dosage form has reduced variability in blood plasma concentrations of celecoxib during the initial one hour after administration. In one measure of reduced variability, at least 50% of patients achieve a C_{0.5} after initial administration of at least 0.9 ng/ml per mg of celecoxib dosed. More preferably, at least 60% of patients achieve a C_{0.5} of at least 0.9 ng/ml per mg of celecoxib dosed, and even more preferably at least 65% of patients achieve a C_{0.5} of at least 0.9 ng/ml per mg of celecoxib dosed.

Another measure of reduced variability is the percentage of patients that achieve a target blood plasma concentration of celecoxib within the first hour after initial administration. In one aspect, the dosage form provides that at least 85% of patients have a C₁ of at least 1.2 ng/ml per mg of celecoxib dosed. Preferably, the dosage form provides that at least 85% of the patients have a C₁ of at least 1.6 ng/mL per mg of celecoxib dosed. In another aspect, the dosage form provides that at least 85% of patients have a C₁ of no greater than 10 ng/ml per mg of celecoxib dosed.

In another measure of reduced variability within the first hour after administration, the dosage form provides a coefficient of variation in C₁ of less than 70%. Coefficient of variation is simply the standard deviation divided by the mean and is a standard statistical measure well known to those skilled in the art. More preferably, the dosage form provides a coefficient of variation in C₁ of less than 60%, and even more preferably provides a coefficient of variation in C₁ of less than 50%.

The dosage forms also provide minimum blood plasma concentrations of celecoxib twelve hours after initial administration relative to the amount of celecoxib in the dosage form. In one embodiment, the dosage form provides a mean blood plasma concentration of celecoxib 12 hours after initial administration (C₁₂) of at least about 0.6 ng/ml per mg of celecoxib dosed. More preferably, the dosage form provides a C₁₂ of at least about 0.65 ng/ml per mg of celecoxib dosed, and even more preferably provides a C₁₂ of at least about 0.7 ng/ml per mg of celecoxib dosed.

The dosage form also provides good exposure relative to the amount of celecoxib in the dosage form. In one aspect, the dosage form provides a mean area under the blood plasma concentration versus time curve for the 12 hour period following administration (AUC₁₂) of at least 19 ng-hr/mL per mg of celecoxib dosed. For example, a dosage form comprising 140 mg of celecoxib that provides an “AUC₁₂ of at least 19 ng-hr/mL per mg of celecoxib dosed” would provide an AUC₁₂ of at least 2,660 ng-hr/mL. Preferably, the dosage form provides an AUC₁₂ of at least 21 ng-hr/mL per mg of celecoxib dosed, and even more preferably at least 23 ng-hr/mL per mg of celecoxib dosed.

The dosage forms also limit the maximum amount of celecoxib in the blood relative to the amount of celecoxib in the dosage form. In one aspect the mean maximum blood plasma concentration of celecoxib (Cₘₐₓ) is less than 4.9 ng/ml per mg of celecoxib dosed. Preferably, the Cₘₐₓ is less than 4.5 ng/ml per mg of celecoxib dosed, and even more preferably less than 4 ng/ml per mg of celecoxib dosed.

One useful measure of the efficacy of the dosage form is the ratio of C₁ to C₁₂. In one embodiment, the ratio of C₁ to C₁₂ is less than 4. Limiting the ratio of C₁ to C₁₂ ensures that a sufficient amount of celecoxib is released and absorbed over a sustained period of time so that pain relief is provided over several hours. Preferably, the ratio of C₁ to C₁₂ is less than 3.5, and even more preferably is less than 3. In addition, to provide rapid pain relief, the ratio of C₁ to C₁₂ should not be too low. Thus, in one embodiment, the ratio of C₁ to C₁₂ is at least 1.0. In another embodiment, the ratio of C₁ to C₁₂ ranges from 1 to 4.

Another useful measure of the efficacy of the dosage form is the ratio of Cₘₐₓ to C₁₂. In one embodiment, the ratio of Cₘₐₓ to C₁₂ is less than 6. Limiting the ratio of Cₘₐₓ to C₁₂ ensures that a sufficient amount of celecoxib is released and absorbed over a sustained period of time so that pain relief is provided over several hours. Preferably, the ratio of Cₘₐₓ to C₁₂ is less than 5.5, and more preferably the ratio of Cₘₐₓ to C₁₂ is less than 5.0. In addition, to provide rapid pain relief, the ratio of Cₘₐₓ to C₁₂ should not be too low. Thus, in one embodiment, the ratio of Cₘₐₓ to C₁₂ is at least 3. In another embodiment, the ratio of Cₘₐₓ to C₁₂ ranges from 3 to 6.

A study to measure the concentration of celecoxib in the blood plasma after initial administration may be conducted using conventional methods for making such a determination. The study should include at least 12 patients in order to measure mean values for C_{0.5}, C₁, C₁₂, Cₘₐₓ and AUC₁₂. The study should be conducted in the fasted state. Prior to the initial administration of celecoxib, the patient has not been administered celecoxib for a sufficient length of time so that the patient's blood plasma concentration of celecoxib prior to administration of the dosage form is below the detectable limit. Blood plasma samples are taken at a sufficient number of time points to determine C_{0.5}, C₁, C₁₂, Cₘₐₓ and AUC₁₂.

Expected concentrations of celecoxib in the blood plasma provided by the dosage forms may also be calculated by pharmacokinetic modeling. A description of a suitable pharmacokinetic model is presented in the Examples hereinafter.

In a second aspect, a pharmaceutical dosage form comprises celecoxib and a pharmaceutically acceptable carrier. The formulation when initially administered orally to at least 12 human patients in a crossover study, provides:

(a) a C_{0.5} of at least 2.8 fold that provided by a control capsule;

(b) a C₁₂ of at least 1.3 fold that provided by the control capsule;

(c) an AUC₁₂ of at least 1.7 fold that provided by the control capsule; and

(d) a Cₘₐₓ of no greater than 2.6 fold that provided by the control capsule;

wherein the control capsule consists of the same amount of celecoxib as the dosage form, but wherein the celecoxib is in crystalline form, and the control capsule further contains lactose, sodium lauryl sulfate, povidone, crosscarmellose sodium and magnesium stearate.

By “control capsule” is meant the commercially available CELEBREX™ capsules for oral administration manufactured by Pfizer, Inc. containing the same amount of active celecoxib in milled, crystalline form. CELEBREX™ capsules contain celecoxib, lactose, sodium lauryl sulfate, povidone, crosscarmellose sodium and magnesium stearate. A control capsule with 200 mg celecoxib is prepared as follows:

| Ingredient | mg | wt % |
|---|---|---|
| celecoxib, milled | 200.00 | 74.07 |
| lactose, monohydrate | 49.75 | 18.43 |
| sodium lauryl sulfate | 8.10 | 3.00 |
| povidone, K29-32 | 6.75 | 2.50 |
| croscarmellose sodium | 2.70 | 1.00 |
| magnesium stearate | 2.70 | 1.00 |
| Total | 270 | 100.00 |

Control capsules with different amounts of celecoxib may be prepared by adjusting the relative amounts of inert carriers. The ingredients are mixed and filled into a gelatin capsule.

In order to provide rapid pain relief, the dosage form provides higher blood levels of celecoxib within the first hour after the initial administration of the dosage form relative to the control capsule. In one embodiment, the dosage form provides a C_{0.5} of at least 2.8 fold that provided by the control capsule. For example, if the control capsule provides a C_{0.5} of 35 ng/ml, then the dosage form provides a C_{0.5} of at least about 98 ng/ml. Preferably, the dosage form provides a C_{0.5} of at least about 3 fold that provided by the control capsule, more preferably at least about 3.5 fold that provided by the control capsule, and even more preferably at least 4 fold that provided by the control capsule.

In another embodiment, the dosage form provides a C₁ of at least 1.5 fold that provided by the control capsule. Preferably, the dosage form provides a C₁ of at least about 2 fold that provided by the control capsule, and more preferably at least about 2.5 fold that provided by the control capsule.

The dosage forms also provide higher blood plasma concentrations of celecoxib at twelve hours after administration relative to the control capsule. In one embodiment, the dosage form provides a C₁₂ of at least 1.3 fold that provided by the control capsule. Preferably, the dosage form provides a C₁₂ of at least 1.4 fold that provided by the control capsule, and more preferably provides a C₁₂ of at least 1.5 fold that provided by the control capsule.

The dosage form also provides higher exposure relative to the control capsule. In one aspect, the dosage form provides an AUC₁₂ of at least 1.7 fold that provided by the control capsule. Preferably, the dosage form provides an AUC₁₂ of at least 2 fold that provided by the control, and more preferably an AUC₁₂ of at least 2.2 fold that provided by the control.

The dosage form also limits the maximum amount of celecoxib in the blood relative to the control capsule. The dosage form provides a Cₘₐₓ of no greater than 2.6 fold that provided by the control capsule. Preferably, the dosage form provides a Cₘₐₓ that is no greater than 2.4 fold that provided by the control capsule, and more preferably no greater than 2.2 fold that provided by the control capsule.

The relative values for C_{0.5}, C₁, C₁₂, and AUC₁₂ compared with the control capsule may be determined in a clinical study in humans using conventional methods for making such a determination. An in vivo test, such as a crossover study, may be used to determine the relative values of C_{0.5}, C₁, C₁₂, and AUC₁₂ provided by the dosage form compared with the control capsule containing the same amount of active celecoxib. In an in vivo crossover study a test dosage form is dosed to half a group of test subjects and, after an appropriate washout period (e.g., one week) the same subjects are dosed with the control capsule that consists of an equivalent quantity of celecoxib. The other half of the group is dosed with the control capsule first, followed by the test dosage form. Preferably, the test/control ratios are determined for each subject, and then the ratios are averaged over all subjects in the study. In vivo determinations of AUC can be made by plotting the serum or plasma concentration of drug along the ordinate (y-axis) against time along the abscissa x-axis). Methods for determining the AUCs and the relative bioavailability of a dosage form are well known in the art. (The calculation of an AUC is a well-known procedure in the pharmaceutical arts and is described, for example, in Welling, “Pharmacokinetics Processes and Mathematics,” ACS Monograph 185 (1986)).

Expected relative values of C_{0.5}, C₁, C₁₂, and AUC₁₂ provided by the dosage form compared with the control capsule may also be determined through pharmacokinetic modeling, as described in the Examples.

In order to achieve the blood levels of celecoxib described above, it is desired that the dosage form release celecoxib in a controlled manner. The following in vitro test may be used to determine if a dosage form is within the scope of the invention. The dosage form is tested in a USP Type II dissolution test at 37° C. with baskets at 100 rpm in 1000 mL of 50 mM sodium phosphate adjusted to pH 6.8 containing 2% (weight/volume) sodium dodecyl sulfate.

In one embodiment, the dosage form releases celecoxib as follows:

10 wt % to 35 wt % of the celecoxib in the dosage form at half hour after administration (in other words, at one half hour after administration, the dosage form would have released from 10 wt % to 35 wt % of the total amount of celecoxib in the dosage form);

15 wt % to 45 wt % at 1 hour after administration;

25 wt % to 60 wt % at 2 hours after administration;

40 wt % to 100 wt % at 5 hours after administration; and

65 wt % to 100 wt % at 12 hours after administration.

In another embodiment, the dosage form releases celecoxib as follows:

15 wt % to 35 wt % of the celecoxib in the dosage form at half hour after administration;

25 wt % to 40 wt % at 1 hour after administration;

40 wt % to 55 wt % at 2 hours after administration;

65 wt % to 95 wt % at 5 hours after administration; and

90 wt % to 100 wt % at 12 hours after administration.

In another embodiment, the dosage form releases celecoxib as follows:

10 wt % to 25 wt % of the celecoxib in the dosage form at half hour after administration;

20 wt % to 35 wt % at 1 hour after administration;

30 wt % to 45 wt % at 2 hours after administration;

50 wt % to 70 wt % at 5 hours after administration; and

80 wt % to 100 wt % at 12 hours after administration.

In another embodiment, the dosage form releases celecoxib as follows:

10 wt % to 25 wt % of the celecoxib in the dosage form at half hour after administration;

15 wt % to 30 wt % at 1 hour after administration;

25 wt % to 40 wt % at 2 hours after administration;

45 wt % to 60 wt % at 5 hours after administration; and

70 wt % to 90 wt % at 12 hours after administration.

The dosage form contains celecoxib in a solubility-improved form. By “solubility-improved form” is meant that the celecoxib is in a form such that it provides higher concentrations of dissolved drug in a use environment relative to a control composition consisting essentially of celecoxib in bulk crystalline form. As used herein, a “use environment” can be either the in vivo environment, such as the gastrointestinal tract of an animal, particularly a human, or the in vitro environment of a test solution, such as phosphate buffered saline (PBS) solution, Model Fasted Duodenal (MFD) solution, simulated gastric buffer solution, or a simulated intestinal buffer solution.

In one embodiment, the solubility-improved form of celecoxib is celecoxib in amorphous form. Preferably, at least 90 wt % of the celecoxib is amorphous. By “amorphous” is meant simply that the celecoxib is in a non-crystalline state. Amounts of crystalline celecoxib may be measured by Powder X-Ray Diffraction (PXRD), Scanning Electron Microscope (SEM) analysis, differential scanning calorimetry (DSC), or any other standard quantitative measurement.

The amorphous form of celecoxib may be in any form in which celecoxib is amorphous. In a preferred embodiment, celecoxib is in the form of a molecular dispersion of amorphous celecoxib in a polymer. By “molecular dispersion” is meant a solid material in which the amorphous drug and the polymer are dispersed throughout one another at the molecular level. Such molecular dispersions are sometimes referred to as amorphous solid solutions. The term “molecular dispersion” is intended to include both amorphous solid solutions that are thermodynamically stable, wherein the drug is present at less than the solubility limit of the drug in the polymer, as well as amorphous solid solutions wherein the drug is present in excess of the solubility limit of the drug in the polymer. Note that a molecular dispersion is different than a simple physical mixture, which consists of particles of amorphous or crystalline celecoxib mixed or blended with particles of polymer.

The polymer used in the molecular dispersion may be any pharmaceutically acceptable polymer. The term “polymer” is used conventionally, meaning a compound that is made of monomers connected together to form a larger molecule. A polymer generally consists of at least about 20 monomers connected together. Thus, the molecular weight of the polymer generally will be about 2000 daltons or more. The polymer should be inert, in the sense that it does not chemically react with the celecoxib in an adverse manner, and should be pharmaceutically acceptable. Exemplary polymers include hydroxypropyl methyl cellulose acetate succinate (HPMCAS), hydroxypropyl methyl cellulose phthalate (HPMCP), hydroxypropyl methyl cellulose (HPMC), cellulose acetate phthalate (CAP), cellulose acetate trimellitate (CAT), carboxymethyl ethylcellulose (CMEC), poloxamers (also known as polyoxyethylene-polyoxypropylene block copolymers), polyvinyl pyrrolidone (PVP), and mixtures thereof.

In one embodiment, the polymer is HPMCAS. HPMCAS is currently commercially available from Shin-Etsu Chemical (Tokyo, Japan), known by the trade name “AQOAT.” Shin-Etsu manufactures three grades of AQOAT that have different combinations of substituent levels to provide enteric protection at various pH levels. The AS-LF and AS-LG grades (the “F” standing for fine and the “G” standing for granular) provide enteric protection up to a pH of about 5.5. The AS-MF and AS-MG grades provide enteric protection up to a pH of about 6.0, while the AS-HF and AS-HG grades provide enteric protection up to a pH of about 6.8.

A preferred grade of HPMCAS is the L grade, having a methoxyl content of from 20 to 24 wt %, a hydroxypropoxyl content of from 5 to 9 wt %, an acetyl content of from 5 to 9 wt %, and a succinoyl content of from 14 to 18 wt %.

In another embodiment, the dispersion polymer is HPMC. HPMC is available under the trade name METHOCEL™ from Dow Chemical Co. A preferred grade of HPMC is the E3 Prem LV grade available from Dow Chemical. This product has a methoxyl content of 28 to 30 wt %, and a hydroxypropyl content of 7 to 12 wt %. The viscosity of a 2 wt % solution of METHOCEL E3 Prem LV in water ranges from 2.4 to 3.6 cps.

The celecoxib and polymer are collectively present in the molecular dispersion in an amount ranging from 80 wt % to 100 wt %. Preferably, the celecoxib and polymer collectively constitute at least 90 wt %, more preferably at least 95 wt % of the molecular dispersion. In one embodiment, the molecular dispersion consists essentially of celecoxib and the polymer. By “consist essentially of” is meant that the molecular dispersion contains less than 1 wt % of any other excipients.

The amount of celecoxib in the molecular dispersion may range from 0.1 wt % to 90 wt %. Preferably the amount of celecoxib in the molecular dispersion ranges from about 15 wt % to about 85 wt %, more preferably from about 25 wt % to about 75 wt %, even more preferably from about 40 wt % to about 60 wt %.

The amount of polymer in the molecular dispersion may range from 10 wt % to 99.9 wt %. Preferably, the amount of polymer ranges from 15 wt % to 85 wt %, more preferably from 25 wt % to 75 wt %, and even more preferably from 40 wt % to 60 wt %.

Preferred embodiments of molecular dispersions have the following amounts of celecoxib and polymer:

25 to 75 wt %, preferably 40 to 60 wt % celecoxib; and

25 to 75 wt %, preferably 40 to 60 wt % polymer.

Molecular dispersions of celecoxib and polymers may be made according to any known method. One preferred method is a melt extrusion method, in which the celecoxib and polymer are heated and extruded together.

In another preferred method, the celecoxib and polymer are dissolved in a common solvent, and the solvent is rapidly removed. Exemplary solvent methods include spray drying and spray granulating.

Alternatively, celecoxib may be adsorbed in amorphous form on a solid substrate. In this embodiment, the amorphous celecoxib may be adsorbed to an inorganic oxide, such as silicon dioxide, with or without a polymer.

In another embodiment, celecoxib may be in the form of nanoparticles as the solubility-improved form. By “nanoparticles” is meant a plurality of small particles in which the average size of the particles in suspension is less than about 500 nm. By “average size” is meant the effective cumulant diameter as measured by dynamic light scattering, using for example, Brookhaven Instruments' 90Plus particle sizing instrument. By “size” is meant the diameter for spherical particles, or the maximum diameter for non-spherical particles. Preferably, the average size of the nanoparticles is less than 400 nm, more preferably less than 300 nm, more preferably less than 200 nm, more preferably less than 150 nm, and most preferably less than 100 nm.

In one embodiment, the nanoparticles are in the form of crystalline drug particles. Examples of such nanoparticles are further described in U.S. Pat. No. 5,145,684. The nanoparticles of the drug can be prepared using any known method for preparing nanoparticles. One method comprises suspending celecoxib in a liquid dispersion medium and applying mechanical means in the presence of grinding media to reduce the particle size of the drug substance to the effective average particle size. The particles can be reduced in size in the presence of a surface modifier. Alternatively, the particles can be contacted with a surface modifier after attrition. Other alternative methods for forming nanoparticles are described in U.S. Pat. No. 5,560,932, and U.S. Pat. No. 5,874,029.

In another embodiment, the nanoparticles are in the form of drug and polymer nanoparticles. The nanoparticles comprise the drug, a polymer, and optional surface stabilizers. At least 90 wt % of the drug in the nanoparticles is amorphous.

Preferred polymers for use in the drug/polymer nanoparticles are non-ionizable, poorly water soluble polymers. In one embodiment, the polymer is selected from the group consisting of methylcellulose, ethylcellulose, propylcellulose, butylcellulose, cellulose acetate, cellulose propionate, cellulose butyrate, cellulose acetate butyrate, cellulose acetate propionate, methyl cellulose acetate, methyl cellulose propionate, methyl cellulose butyrate, ethyl cellulose acetate, ethyl cellulose propionate, ethyl cellulose butyrate, low-substituted hydroxypropyl cellulose, hydroxypropyl methylcellulose acetate, hydroxypropyl methylcellulose propionate, hydroxypropyl methylcellulose butyrate, poly(lactide), poly(glycolide), poly(ε-caprolactone), poly(lactide-co-glycolide), poly(lactide-co-ε-caprolactone), poly(ethylene oxide-co-ε-caprolactone), poly(ethylene oxide-co-lactide), poly(ethylene oxide-co-lactide-co-glycolide), poly(isobutyl)cyanoacrylate, and poly(hexyl)cyanoacrylate.

In a preferred embodiment, the polymer is selected from the group consisting of ethylcellulose and poly(ethylene oxide-co-c-caprolactone). Most preferably, the polymer is ethylcellulose.

Exemplary surface stabilizers include casein, caseinates, polyvinyl pyrrolidone (PVP), polyoxyethylene alkyl ethers, polyoxyethylene stearates, polyoxyethylene castor oil derivatives, poly(ethylene oxide-propylene oxide) (also known as poloxamers), tragacanth, gelatin, polyethylene glycol, bile salts (such as salts of dihydroxy cholic acids, including sodium and potassium salts of cholic acid, glycocholic acid, and taurocholic acid), phospholipids (such as phosphatidyl cholines, including 1,2-diacylphosphatidylcholine also referred to as PPC or lecithin), sodium dodecylsulfate (also known as sodium lauryl sulfate), benzalkonium chloride, sorbitan esters, polyoxyethylene alkyl ethers, polyoxyethylene castor oil derivatives, polyoxyethylene sorbitan fatty acid esters (polysorbates), polyoxyethylene stearates, triethanolamine, sodium docusate, sodium stearyl fumarate, sodium cyclamate, and mixtures and pharmaceutically acceptable forms thereof.

The celecoxib, polymer, and the optional surface stabilizer are collectively present in the nanoparticle in an amount ranging from 80 wt % to 100 wt %. Preferably, the celecoxib, polymer, and the optional surface stabilizer collectively constitute at least 90 wt %, more preferably at least 95 wt % of the nanoparticle. In one embodiment, the nanoparticles consist essentially of the celecoxib, the non-ionizable polymer, and the optional surface stabilizer. By “consist essentially of” is meant that the nanoparticle contains less than 1 wt % of any other excipients and that any such excipients have no affect on the performance or properties of the nanoparticle.

The amount of celecoxib in the nanoparticle may range from 0.1 wt % to 90 wt %. Preferably the amount of celecoxib in the nanoparticle ranges from about 1 wt % to about 85 wt %, more preferably from about 5 wt % to about 80 wt %, even more preferably from about 10 wt % to about 75 wt %, and most preferably from about 20 wt % to about 50 wt %.

The amount of polymer may range from 10 wt % to 99.9 wt %. The physical stability of the celecoxib in the nanoparticle tends to improve with increasing amounts of the poorly aqueous soluble non-ionizable polymer. Accordingly, it is preferred that the amount of polymer in the nanoparticle is at least 15 wt %, more preferably at least 20 wt %, and most preferably at least 25 wt %. However, too much polymer will lead to a low loading of celecoxib in the nanoparticle. Thus, it is preferred that the amount of polymer in the nanoparticle is 75% or less, and most preferably 70 wt % or less.

In one embodiment, the nanoparticles further comprise a surface stabilizer. When a surface stabilizer is present, the amount may range from 0.1 wt % to 50 wt % of the nanoparticle. The surface stabilizer acts to reduce or prevent aggregation or flocculation of the nanoparticles in an aqueous suspension, resulting in nanoparticles with improved stability. Generally, lower concentrations of surface stabilizer are preferred. Thus, preferably the surface stabilizer constitutes about 45 wt % or less, more preferably about 40 wt % or less, and most preferably about 35 wt % or less the total mass of the nanoparticles.

Preferred embodiments of nanoparticles have the following amounts of celecoxib, polymer, and optional surface stabilizer:

10 to 75 wt %, preferably 20 to 50 wt % celecoxib;

20 to 75 wt %, preferably 25 to 70 wt % polymer; and

0 to 50 wt %, preferably 1 to 40 wt % optional surface stabilizer.

In one embodiment, the nanoparticles comprise at least 30 wt % celecoxib and at least 30 wt % of polymer.

In another embodiment, the nanoparticles comprise 35 to 40 wt % celecoxib and 35 to 40 wt % of polymer.

The nanoparticles may be formed by any process that results in formation of nanoparticles of the celecoxib and a non-ionizable polymer. The celecoxib used to form the nanoparticles may be in a crystalline or non-crystalline form; however, at least 90 wt % of the celecoxib in the resulting nanoparticles is in amorphous or non-crystalline form.

One process for forming nanoparticles is an emulsification process. In this process, the celecoxib and polymer are dissolved in an organic solvent that is immiscible with an aqueous solution in which the celecoxib and polymer are poorly soluble, forming an organic solution. Once the organic solution is formed, it is then mixed with the aqueous solution and homogenized to form an emulsion of fine droplets of the water immiscible solvent distributed throughout the aqueous phase. The organic solvent is removed to form a suspension of solid nanoparticles, the nanoparticles comprising celecoxib and polymer.

An alternative process to form the nanoparticles is a precipitation process. In this process, the celecoxib and polymer are first dissolved in an organic solvent that is miscible with an aqueous solution in which the celecoxib and polymer are poorly soluble to form an organic solution. The organic solution is mixed with the aqueous solution causing the nanoparticles to precipitate.

Once the nanoparticle suspension is made, a portion of the organic solvent may be removed from the suspension using methods known in the art. Exemplary processes for removing the organic solvent include evaporation, extraction, diafiltration, pervaporation, vapor permeation, distillation, and filtration.

When isolating the nanoparticles in solid form, it is often desirable to include a resuspending material into the suspension of nanoparticles prior to removal of the liquids. The resuspending material functions to help slow or prevent agglomeration of the nanoparticles as the liquids are being removed, as well as to help re-suspend the nanoparticles when the solid composition is added to an aqueous solution (e.g., an aqueous environment of use). The resuspending material is preferably pharmaceutically acceptable and water soluble. Examples of resuspending materials include polyvinyl pyrrolidone (PVP), trehalose, hydroxypropyl methyl cellulose (HPMC), hydroxypropyl cellulose (HPC), casein, caseinate, albumin, gelatin, acacia, lactose, mannitol, pharmaceutically acceptable forms thereof, and other resuspending materials known in the art.

Several methods, such as an in vitro dissolution test may be used to determine if a form of celecoxib is a solubility-improved form and the degree of solubility improvement. When the solubility-improved form is larger than 1 micron in size, an in vitro dissolution test may be performed by adding the solubility-improved form of celecoxib to a dissolution test media, such as phosphate buffered saline (PBS) solution or model fasted duodenal (MFD) solution. An appropriate PBS solution is an aqueous solution comprising 20 mM Na₂HPO₄, 47 mM KH₂PO₄, 87 mM NaCl, and 0.2 mM KCl, adjusted to pH 6.5 with NaOH. An appropriate MFD solution is the same PBS solution wherein there is also present 7.3 mM sodium taurocholic acid and 1.4 mM of 1-palmitoyl-2-oleyl-sn-glycero-3-phosphocholine.

In one method for evaluating whether the form is a solubility-improved form, the solubility-improved form of celecoxib when tested in an in vitro dissolution test meets at least one, and preferably both, of the following conditions. The first condition is that the solubility-improved form provides a higher maximum dissolved drug concentration (MDC) of celecoxib in the in vitro dissolution test relative to a control composition consisting of the crystalline celecoxib in bulk form. That is, once the solubility-improved form is introduced into a use environment, the solubility-improved form provides a higher aqueous concentration of dissolved celecoxib relative to the control composition. It is important to note that the solubility-improved form is dissolution tested independently of the dosage form so that the sustained release means do not interfere with evaluation of the degree of solubility improvement. Preferably, the solubility-improved form provides an MDC of celecoxib in aqueous solution that is at least 1.25-fold that of the control composition, more preferably at least 2-fold, and most preferably at least 3-fold. For example, if the MDC provided by the test composition is 22 μg/ml, and the MDC provided by the control composition is 2 μg/ml, the solubility-improved form provides an MDC that is 11-fold that provided by the control composition.

The second condition is that the solubility-improved form provides a higher dissolution area under the concentration versus time curve (DAUC) of dissolved celecoxib in the in vitro dissolution test relative to the control composition. More specifically, in the in vitro use environment, the solubility-improved form provides a DAUC for the 90-minute period following introduction to the use environment that is at least 1.25-fold that of the control composition described above. Preferably, the DAUC provided by the composition is at least 2-fold, more preferably at least 3-fold that of the control composition.

An in vitro test to evaluate enhanced celecoxib concentration in aqueous solution can be conducted by (1) adding with agitation a sufficient quantity of control composition, that is, the crystalline celecoxib alone, to the in vitro test medium, such as an MFD or PBS solution to achieve equilibrium concentration of celecoxib; (2) in a separate test, adding with agitation a sufficient quantity of test composition (e.g., the solubility-improved form) in the same test medium, such that if all celecoxib dissolved, the theoretical concentration of celecoxib would exceed the equilibrium concentration provided by crystalline celecoxib by a factor of at least 2, and preferably by a factor of at least 10; and (3) comparing the measured MDC and/or aqueous DAUC of the test composition in the test medium with the equilibrium concentration, and/or with the aqueous DAUC of the control composition.

The concentration of dissolved celecoxib is typically measured as a function of time by sampling the test medium and plotting celecoxib concentration in the test medium vs. time so that the MDC can be ascertained. The MDC is taken to be the maximum value of dissolved celecoxib measured over the duration of the test. The aqueous DAUC is calculated by integrating the concentration versus time curve over the 90-minute time period following introduction of the composition into the aqueous use environment.

To avoid large drug particulates that would give an erroneous determination, the test solution is either filtered or centrifuged. “Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred solubility-improved forms. It should be recognized that this definition of “dissolved drug” encompasses not only monomeric solvated drug molecules but also a wide range of species such as polymer/drug assemblies that have submicron dimensions such as drug aggregates, aggregates of mixtures of polymer and drug, micelles, polymeric micelles, colloidal particles or nanocrystals, polymer/drug complexes, and other such drug-containing species that are present in the filtrate or supernatant in the specified dissolution test.

An in vitro membrane permeation test may also be used to determine if a formulation is a solubility-improved form of celecoxib, as described in detail below in the Examples section.

Further details of this membrane permeation test are presented PCT Patent Application No. WO2005095950A1, the disclosure of which is incorporated herein by reference.

In general terms, a typical in vitro membrane permeation test to evaluate enhanced drug concentration can be conducted by providing a drug-permeable membrane between feed and permeate reservoirs, as described in detail in the Examples, then (1) administering a sufficient quantity of test composition (that is, the solubility-improved form of celecoxib) to a feed test medium, such that if all of the drug dissolved, the theoretical concentration of drug would exceed the equilibrium concentration of the drug by a factor of at least 2; (2) separately adding an equivalent amount of control composition (that is, crystalline celecoxib) to an equivalent amount of feed test medium; (3) measuring the flux of drug across the membrane from the feed to the permeate reservoir; and (4) determining whether the measured maximum flux of drug provided by the test composition is at least 1.25-fold that provided by the control composition. A solubility-improved form of celecoxib, when administered to an aqueous use environment, provides a maximum flux of drug in the above test that is at least about 1.25-fold the maximum flux provided by the control composition. Preferably, the maximum flux is at least about 1.5-fold, more preferably at least about 2-fold, and most preferably at least about 3-fold that provided by the control composition.

When the solubility-improved form is smaller than 1 micron in size, an in vitro membrane permeation test may be performed as described above, to determine if a form of celecoxib is a solubility-improved form. Alternatively, the amount of “free” drug or solvated drug is measured. By “free” drug is meant drug which is in the form of dissolved drug or present in micelles, but which is not in the solubility-improved form (such as a nanoparticle or drug-polymer aggregate). A drug form is a solubility-improved form if it provides a free drug concentration that is at least 1.25-fold that provided by the control composition (crystalline drug). Preferably, the solubility-improved form provides a free drug concentration that is at least 2-fold, and more preferably at least 3-fold that of the control composition.

Several procedures can be used to measure free drug for forms smaller than 1 micron in size. In the filtration procedure, a sample of the drug form is equilibrated in an aqueous receptor solution, such as water, PBS, or MFD solution by stirring. An aliquot of ˜300 μL is withdrawn and placed into a microcentrifuge tube fitted with a 100,000 molecular weight (MW) cutoff filter (regenerated cellulose). The tube is spun at 13,000 rpm for 3 minutes, and the filtrate solution is collected. The filtrate solution contains only drug that is dissolved, as the drug form cannot pass through the MW cutoff filter. The drug concentration in the filtrate is analyzed by HPLC.

Alternatively, free drug can be measured with nuclear magnetic resonance (NMR). In this method, a sample of the drug form is equilibrated in an NMR tube with a buffered deuterium oxide solution. A specified amount of a reference standard (a suitable reference standard is trifluoroacetic acid) is also added to the sample, such that the final concentration of the standard in the tube is known. A fluorine NMR spectrum is then acquired, and the integration of the drug peak(s) is compared to that of the reference standard to determine the actual dissolved drug concentration. Because NMR is sensitive only to materials in the solution state or in micelles, only the drug that is not sequestered in particles is measured by this method.

The dosage form of the present invention may be any dosage form capable of providing the blood levels of celecoxib or in vitro release rates described above. In one embodiment, the dosage form comprises (a) an immediate release (IR) portion and (b) a sustained release (SR) portion.

By “immediate release portion” is meant that 90 wt % of the celecoxib in the immediate release portion is released within two hours or less following administration to a gastric use environment, preferably within one hour or less following administration. “Administration” to a use environment means, where the in vivo use environment is the GI tract, delivery by ingestion or swallowing or other such means to deliver the dosage form. Where the use environment is in vitro, “administration” refers to placement or delivery of the dosage form to the in vitro test medium. Immediate release of drug may be accomplished by any means known in the pharmaceutical arts, including immediate release coatings, immediate release layers, and immediate release multiparticulates or granules. Exemplary dosage forms include tablets; caplets, capsules; powders or granules; chewable tablets; unit dose packets, sometimes referred to in the art as “sachets” or “oral powders for constitution” (OPC); syrups; and suspensions.

In one embodiment, the immediate release portion is a layer comprising the solubility-improved form of celecoxib and other excipients or carriers. When the solubility-improved form is a molecular dispersion, the molecular dispersion may be present in the layer in an amount of from 40 to 80 wt %, more preferably 50 to 70 wt % of the layer.

The immediate release portion may comprise a disintegrant. Examples of disintegrants include sodium starch glycolate, sodium carboxymethyl cellulose, calcium carboxymethyl cellulose, croscarmellose sodium, crospovidone, polyvinyl pyrrolidone, methyl cellulose, microcrystalline cellulose, powdered cellulose, lower alkyl-substituted hydroxypropyl cellulose, polacrilin potassium, starch, pregelatinized starch, sodium alginate, and mixtures thereof. Generally, the disintegrant will comprise from 1 wt % to 15 wt %, preferably from 2 wt % to 10 wt % of the layer.

The immediate release portion may also include a porosigen. A “porosigen” is a material that, when present in the formulation containing the molecular dispersion, leads to a high porosity and high strength following compression of the blend into a tablet. Examples of porosigens include acacia, calcium carbonate, calcium sulfate, calcium sulfate dihydrate, compressible sugar, dibasic calcium phosphate (anhydrous and dihydrate), tribasic calcium phosphate, monobasic sodium phosphate, dibasic sodium phosphate, lactose, magnesium oxide, magnesium carbonate, silicon dioxide, magnesium aluminum silicate, maltodextrin, mannitol, methyl cellulose, microcrystalline cellulose, sorbitol, sucrose, xylitol and mixtures thereof. Of these, microcrystalline cellulose, both forms of dibasic calcium phosphate (anhydrous and dihydrate), and mixtures thereof are preferred. Generally, the porosigen will comprise from 5 to 70 wt %, and preferably from 10 to 50 wt % of the layer.

Other conventional formulation excipients may be employed in the dosage forms of the invention, including those excipients well known in the art, e.g., as described in Remington: The Science and Practice of Pharmacy (20th ed. 2000). Generally, excipients such as surfactants, pH modifiers, fillers, matrix materials, complexing agents, solubilizers, pigments, lubricants, glidants, flavorants, and so forth may be used for customary purposes and in typical amounts without adversely affecting the properties of the compositions.

Examples of matrix materials, fillers, or diluents include lactose, mannitol, xylitol, dextrose, sucrose, sorbitol, compressible sugar, microcrystalline cellulose, powdered cellulose, starch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers, polyethylene oxide, hydroxypropyl methyl cellulose and mixtures thereof.

In one embodiment, the immediate release portion comprises:

30 to 80 wt %, preferably 30 to 65 wt %, more preferably 35 to 45 wt % molecular dispersion;

1 to 15 wt %, more preferably 2 to 10 wt % disintegrant;

20 to 60 wt %, more preferably 30 to 50 wt % diluent; and

0.05 to 2 wt % lubricant.

In another embodiment, the immediate release portion comprises

35 wt % to 45 wt % molecular dispersion;

30 wt % to 40 wt % microcrystalline cellulose;

2 wt % to 7 wt % croscarmellose sodium; and

0.05 wt % to 1 wt % magnesium stearate.

The oral dosage forms also preferably comprise a sustained-release portion. The sustained release portion comprises the solubility-improved form of celecoxib and other carriers and excipients. “Sustained release” means that the sustained release portion releases no greater than about 90 wt % of the celecoxib in the sustained release portion during the first two hours after administration to a use environment. Thus the dosage form may release celecoxib gradually and continuously over a release period, in a pulsed manner, or in a delayed manner. The sustained release portion can be any dosage form or device known in the pharmaceutical arts that allows delivery of a drug in a sustained manner. Exemplary dosage forms include erodible and non-erodible matrix sustained-release dosage forms, osmotic sustained-release dosage forms, multiparticulates, and enteric coated cores.

In one embodiment, the solubility-improved form of celecoxib is incorporated into an erodible or non-erodible polymeric matrix sustained release layer. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous use environment, the erodible polymeric matrix imbibes water and forms an aqueous-swollen gel or “matrix” that entraps the celecoxib. The aqueous-swollen matrix gradually erodes, swells, disintegrates, disperses or dissolves in the environment of use, thereby controlling the release of celecoxib to the environment of use. Examples of such dosage forms are well known in the art. See, for example, Remington: The Science and Practice of Pharmacy, 20^{th} Edition, 2000.

A key ingredient of the water-swollen matrix is the water-swellable, erodible, or soluble polymer, which may generally be described as an osmopolymer, hydrogel or water-swellable polymer. Such polymers may be linear, branched, or crosslinked. They may be homopolymers or copolymers. Exemplary polymers include naturally occurring polysaccharides such as chitin, chitosan, dextran and pullulan; gum agar, gum arabic, gum karaya, locust bean gum, gum tragacanth, carrageenans, gum ghatti, guar gum, xanthan gum and scleroglucan; starches such as dextrin and maltodextrin; hydrophilic colloids such as pectin; phosphatides such as lecithin; alginates such as ammonium alginate, sodium, potassium or calcium alginate, propylene glycol alginate; gelatin; collagen; and cellulosics. By “cellulosics” is meant a cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent.

A preferred class of cellulosics for the erodible matrix comprises aqueous-soluble and aqueous-erodible cellulosics such as ethyl cellulose (EC), methylethyl cellulose (MEC), carboxymethyl cellulose (CMC), carboxymethyl ethylcellulose (CMEC), hydroxyethyl cellulose (HEC), hydroxypropyl cellulose (HPC), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC).

A particularly preferred class of such cellulosics comprises various grades of low viscosity (MW less than or equal to 50,000 daltons) and high viscosity (MW greater than 50,000 daltons) HPMC. Commercially available low viscosity HPMC polymers include the Dow METHOCEL™ series E3, E5, E15LV, E50LV and K100LV, while high viscosity HPMC polymers include E4MCR, E10MCR, K4M, K15M and K100M; especially preferred in this group are the METHOCEL™ K series. Other commercially available types of HPMC include the Shin Etsu METOLOSE™ 90SH series. In one embodiment, the HPMC has a low viscosity, meaning that the viscosity of a 2% (w/v) solution of the HPMC in water is less than about 120 cp. A preferred HPMC is one in which the viscosity of a 2% (w/v) solution of the HPMC in water ranges from 80 to 120 cp (such as METHOCEL™ K100LV).

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl) methacrylate chloride.

The erodible matrix polymer may also contain additives and excipients known in the pharmaceutical arts, including osmopolymers, osmagens, solubility-enhancing or -retarding agents and excipients that promote stability or processing of the dosage form.

Alternatively, the sustained-release portion may comprise a non-erodible matrix. In such dosage forms, celecoxib in a solubility-improved form is distributed in an inert matrix. The drug is released by diffusion through the inert matrix. Examples of materials suitable for the inert matrix include insoluble plastics, such as copolymers of ethylene and vinyl acetate, methyl acrylate-methyl methacrylate copolymers, polyvinyl chloride, and polyethylene; hydrophilic polymers, such as ethyl cellulose, cellulose acetate, and crosslinked polyvinylpyrrolidone (also known as crospovidone); and fatty compounds, such as carnauba wax, microcrystalline wax, and triglycerides. Such dosage forms are described further in Remington: The Science and Practice of Pharmacy, 20^{th} edition (2000).

Other conventional formulation excipients may be employed in the sustained release portion of the invention, including those excipients well known in the art, e.g., as described in Remington: The Science and Practice of Pharmacy, 20th edition (2000). Generally, excipients such as surfactants, pH modifiers, fillers, matrix materials, complexing agents, solubilizers, pigments, lubricants, glidants, flavorants, and so forth may be used for customary purposes and in typical amounts without adversely affecting the properties of the compositions.

Examples of matrix materials, fillers, or diluents include lactose, mannitol, xylitol, dextrose, sucrose, sorbitol, compressible sugar, microcrystalline cellulose, powdered cellulose, starch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers, polyethylene oxide, hydroxypropyl methyl cellulose and mixtures thereof.

In one embodiment, the sustained release portion comprises:

30 to 80 wt %, more preferably 35 to 60 wt % molecular dispersion;

10 to 50 wt %, more preferably 20 to 45 wt % matrix material;

2 to 45 wt %, more preferably 15 to 35 wt % diluent; and

0.05 to 2 wt % lubricant.

In another embodiment, the sustained release portion comprises:

35 wt % to 45 wt % molecular dispersion;

25 wt % to 45 wt % hydroxypropyl methyl cellulose;

15 wt % to 35 wt % lactose; and

0.05 wt % to 1 wt % magnesium stearate

The total amount of celecoxib in the dosage form may range from 10 mg to 400 mg, preferably 20 mg to 300 mg, more preferably 70 mg to 280 mg.

The amount of celecoxib in the immediate release portion may range from 10 mg to 160 mg, preferably 20 mg to 80 mg.

The amount of celecoxib in the sustained release portion may range from 10 mg to 300 mg, preferably from 50 mg to 200 mg.

In one embodiment, the dosage form comprises an immediate release portion and a sustained release portion as follows.

The immediate release portion is from 25 wt % to 60 wt % of the dosage form and comprises:

30 to 80 wt %, more preferably 35 to 60 wt % molecular dispersion of celecoxib and HPMCAS;

1 to 15 wt %, more preferably 2 to 10 wt % disintegrant;

20 to 60 wt %, more preferably 25 to 50 wt % diluent; and

0.05 to 2 wt % lubricant.

The sustained release portion is from 40 wt % to 75 wt % of the dosage form and comprises:

30 to 80 wt %, more preferably 35 to 65 wt % molecular dispersion of celecoxib and HPMCAS;

10 to 50 wt %, more preferably 15 to 45 wt % matrix material;

2 to 40 wt %, more preferably 5 to 30 wt % diluent; and

0.05 to 2 wt % lubricant.

In another embodiment, the dosage form comprises an immediate release portion and a sustained release portion as follows.

The immediate release portion is from 25 wt % to 60 wt % of the dosage form and comprises:

30 to 80 wt %, more preferably 35 to 60 wt % molecular dispersion of celecoxib and HPMC;

1 to 15 wt %, more preferably 2 to 10 wt % disintegrant;

20 to 60 wt %, more preferably 25 to 50 wt % diluent; and

0.05 to 2 wt % lubricant.

The sustained release portion is from 40 wt % to 75 wt % of the dosage form and comprises:

30 to 80 wt %, more preferably 35 to 65 wt % molecular dispersion of celecoxib and HPMC;

10 to 50 wt %, more preferably 15 to 45 wt % matrix material;

2 to 40 wt %, more preferably 5 to 30 wt % diluent; and

0.05 to 2 wt % lubricant.

In another embodiment, the dosage form comprises an immediate release portion and a sustained release portion as follows.

The immediate release portion is from 10 wt % to 60 wt % of the dosage form and comprises:

20 to 80 wt %, more preferably 30 to 70 wt % nanoparticles of celecoxib and ethylcellulose and optional surface stabilizer;

5 to 50 wt %, more preferably 10 to 40 wt % resuspending material;

1 to 15 wt %, more preferably 2 to 10 wt % disintegrant;

10 to 60 wt %, more preferably 20 to 50 wt % diluent; and

0.05 to 2 wt % lubricant.

The sustained release portion is from 40 wt % to 90 wt % of the dosage form and comprises:

20 to 80 wt %, more preferably 30 to 70 wt % nanoparticles of celecoxib and ethylcellulose and optional surface stabilizer;

5 to 50 wt %, more preferably 10 to 40 wt % resuspending material;

10 to 50 wt %, more preferably 15 to 45 wt % matrix material;

2 to 40 wt %, more preferably 5 to 30 wt % diluent; and

0.05 to 2 wt % lubricant.

The dosage forms may be used to treat any indication for which celecoxib may be prescribed. Exemplary indications include for relief of the signs and symptoms of osteoarthritis, for relief of the signs and symptoms of rheumatoid arthritis in adults, for relief of the signs and symptoms of juvenile rheumatoid arthritis in patients 2 years and older, for the relief of signs and symptoms of ankylosing spondylitis, for the management of acute pain in adults, for the treatment of primary dysmenorrheal, and to reduce the number of adenomatous colorectal polyps in familial adenomatous polyposis (FAP), as an adjunct to usual care (e.g., endoscopic surveillance, surgery).

The dosage form may be administered as necessary to treat the indication. Two dosage forms are initially administered orally to the patient to provide initially high blood concentrations of celecoxib. The two initial dosage forms are referred to as the “loading dose.” Following the loading dose, the dosage form may be administered twice daily. Alternatively, other dosing regimens may be followed.

In one embodiment, the loading dose comprises from about 240 mg to about 320 mg celecoxib and provides the following blood levels of celecoxib after initial administration:

C_{0.5} of at least 200 ng/ml, more preferably at least 215 ng/ml, and even more preferably at least 240 ng/ml;

C₁ of at least 460 ng/ml, more preferably at least 500 ng/ml, and even more preferably at least 560 ng/ml;

AUC₁₂ of at least 5,400 ng-hr/mL, more preferably at least 5,800 ng-hr/mL, and even more preferably at least 6,500 ng-hr/mL; and

Cₘₐₓ of less than 1,400 ng/ml, more preferably less than 1,300 ng/ml, and even more preferably less than 1,200 ng/ml.

One of the advantages of the present dosage form is that the dosage form achieves steady state quickly. By “steady state” is meant the state achieved after administration of the dosage form over a sufficient period of time (e.g., from three days to a week) so that the maximum and minimum celecoxib concentrations in the blood have plateaued (that is, reached a relatively constant value). (Of course, reference to administration of a dosage form means dosage forms having the same composition are administered once or twice a day to achieve steady state, and not that a single dosage form is repeatedly administered). In one embodiment, steady state is achieved following the loading dose and five additional administrations of a single dosage form given twice a day. Preferably, steady state is achieved following the loading dose and four additional administrations of a single dosage form given twice a day.

### EXAMPLES

### Preparation of Solubility Improved Forms of Celecoxib

### Solubility-Improved Form 1

A molecular dispersion containing 50 wt % celecoxib and 50 wt % HPMCAS-LG (AQOAT-LG™, Shin Etsu, Tokyo, Japan) (referred to as the “HPMCAS SDD”) was prepared by spray drying using the following procedure. First, 2,515.1 g of celecoxib (99.4 wt % active) and 2,484.9 gm of HPMCAS-LG was dissolved in 45 kg methanol by mixing for about 1 hour to form the spray solution.

The HPMCAS SDD was formed using the following procedure. The spray solution was pumped using a high-pressure pump (a Bran Luebbe, model N-P31) to a spray drier (a Niro type XP Portable Spray-Dryer with a Liquid-Feed Process Vessel) (“PSD-1”), equipped with a pressure nozzle (Schlick 2.0 available from Dusen Schlick GmbH of Untersiemau, Germany). The PSD-1 was equipped with 9-inch and 4-inch chamber extensions. The spray drier was also equipped with a DPH gas disperser for introduction of the drying gas to the spray drying chamber. The spray solution was pumped to the spray drier at about 65 g/min at a pressure of about 412 psi. Drying gas (e.g., nitrogen) was introduced to the spray drier through the DPH lid at a flow rate of about 1875 g/min and at an inlet temperature of about 109° C. The evaporated solvent and wet drying gas exited the spray drier at a temperature of about 55° C.

The HPMCAS SDD formed using the above procedure was post-dried using a Gruenberg convection tray dryer with a powder depth of about 1 cm operating at 40° C. for a minimum of 3 hours. Analysis of the HPMCAS SDD by PXRD showed that more than 95% of the SDD was non-crystalline.

An in vitro dissolution test was used to determine the dissolution performance of the HPMCAS SDD. For this test, a sufficient amount of HPMCAS SDD was added to a microcentrifuge test tube so that the concentration of celecoxib would have been 1000 μgA/mL had all of the compound dissolved. The test was run in duplicate. The test tubes were placed in a 37° c. temperature-controlled chamber, and an MFD solution consisting of 1.8 mL PBS at pH 6.5 and 290 mosm/kg, containing 7.3 mm sodium taurocholic acid and 1.4 mm of 1-palmitoyl-2-oleyl-sn-glycero-3-phosphocholine (0.5 wt % NaTC/POPC) was added to each tube. The samples were mixed with a vortex mixer for one minute, then centrifuged at 13,000 g and 37° C. for one minute. The resulting supernatant solution was then sampled and diluted 1:5 (by volume) with methanol and analyzed by high-performance liquid chromatography (HPLC). HPLC analysis was performed using a Zorbax SB-C₈ column. The mobile phase consisted of 45% 5 mm triethanolamine, adjusted to pH 7.0, and 55% acetonitrile, with a flow rate of 1.5 mL/min. UV absorbance was measured at 254 nm. The contents of each tube were mixed on the vortex mixer and allowed to stand undisturbed at 37° C. until the next sample was taken. Samples were collected at 4, 10, 20, 40, 90, and 1200 minutes.

As a control, the same test was performed with crystalline celecoxib alone, and a sufficient amount of the drug was added so that the drug's concentration would have been 1000 μgA/mL had all of the drug dissolved.

The concentrations of drug obtained in these samples were used to determine the maximum dissolved concentration of drug and the area under the concentration-versus-time curve during the initial 90 minutes (MDC₉₀ and DAUC₉₀, respectively). The results for the HPMCAS SDD and the control are shown in Table 1.

TABLE 1

| | MDC₉₀ | DAUC₉₀ |
|---|---|---|
| Formulation | (μgA/mL) | (min*μgA/mL) |
| HPMCAS SDD | 660 | 50,400 |
| Crystalline celecoxib | 60 | 4100 |

The results show that the HPMCAS SDD consisting of 50 wt % celecoxib and HPMCAS provided concentration-enhancement relative to the crystalline celecoxib alone. The HPMCAS SDD provided an MDC₉₀ that was 11-fold that provided by crystalline drug alone, and a DAUC₉₀ that was 12.3-fold that provided by crystalline drug alone. Thus, the HPMCAS SDD is a solubility-improved form of celecoxib.

### Solubility-Improved Form 2

A molecular dispersion containing 50 wt % celecoxib and 50 wt % HPMC (Methocel™ E3 Prem LV, Dow, Midland, Mich.) (referred to as the “HPMC SDD”) was prepared by spray drying using the following procedure. First, 126 g of celecoxib and 126 gm of HPMC was dissolved in 2609 g methanol and 289.8 g distilled water by mixing for about 1 hour to form the spray solution.

The HPMC SDD was formed using the following procedure. The spray solution was pumped using a high-pressure pump (a Bran Luebbe, model N-P31) to a spray drier (a Niro type XP Portable Spray-Dryer with a Liquid-Feed Process Vessel) (“PSD-1”), equipped with a pressure nozzle (Schlick 1.5 available from Dusen Schlick GmbH of Untersiemau, Germany). The PSD-1 was equipped with 9-inch and 4-inch chamber extensions. The spray drier was also equipped with a DPH gas disperser for introduction of the drying gas to the spray drying chamber. The spray solution was pumped to the spray drier at about 42 g/min at a pressure of about 260 psi. Drying gas (e.g., nitrogen) was introduced to the spray drier through the DPH lid at a flow rate of about 1950 g/min and at an inlet temperature of about 108° C. The evaporated solvent and wet drying gas exited the spray drier at a temperature of about 60° C.

The HPMC SDD formed using the above procedure was post-dried using a Gruenberg convection tray dryer with a powder depth of about 1 cm operating at 40° C. for 24 hours. Analysis of the HPMC SDD by PXRD showed that more than 95% of the SDD was non-crystalline.

An in vitro dissolution test was used to determine the dissolution performance of the HPMC SDD, using the procedures described for the HPMCAS SDD. The concentrations of drug obtained in these samples were used to determine the maximum dissolved concentration of drug and the area under the concentration-versus-time curve during the initial 90 minutes (MDC₉₀ and DAUC₉₀, respectively). The results for the HPMC SDD and the control are shown in Table 1.

TABLE 1

| | MDC₉₀ | DAUC₉₀ |
|---|---|---|
| Formulation | (μgA/mL) | (min*μgA/mL) |
| HPMC SDD | 420 | 30,900 |
| Crystalline celecoxib | 60 | 4100 |

The results show that the HPMC SDD consisting of 50 wt % celecoxib and HPMC provided concentration-enhancement relative to the crystalline celecoxib alone. The HPMC SDD provided an MDC₉₀ that was 7-fold that provided by crystalline drug alone, and a DAUC₉₀ that was 7.5-fold that provided by crystalline drug alone. Thus, the HPMC SDD is a solubility-improved form of celecoxib.

### Solubility-Improved Form 3

A molecular dispersion containing 34.4 wt % celecoxib, 55.6 wt % HPMCAS-LG (AQOAT-LG™, Shin Etsu), and 10 wt % crospovidone was prepared using a hot-melt extrusion process as follows (referred to as the “HME”). First, all of the HPMCAS-LG and crospovidone, and one quarter of the celecoxib were blended in a V blender for 2 minutes. An additional quarter of the celecoxib was then added to the mixture, and the mixture was blended for another 2 minutes, at which time a third quarter of the celecoxib was added and the mixture blended for another 2 minutes. The last quarter of the celecoxib was then added and the mixture was blended for 6 minutes, resulting in the powder feed to the extruder.

The powder feed was fed to a Leistritz ZSE-27 HP 27 mm twin screw extruder (American Leistritz Extruder Corp., Somerville, N.J.). The following screw design was employed, as listed from the feed section downstream.

5-mm Spacer ring
5-mm Spacer ring
5-mm Spacer ring
5-mm Spacer ring
5-mm Spacer ring
5-mm Spacer ring
5-mm Spacer ring
5-mm Spacer ring
GFA-2-40-30
GFA-2-40-90
GFA-2-40-90
GFA-2-40-90
KBS-2-30-30°-RE (forward convey)
GFA-2-40-90
GFA-2-40-30
GFA-2-40-30
GFA-2-20-60
KBS-2-30-60°-RE (forward convey)
GFA-2-20-60
GFA-2-20-30
GFA-2-20-60
GFA-2-15-30
Screw Tip

A K-Tron (K-Tron International, Inc, Pitman, N.J.) Feeder (K PH ML-KT20) was used to feed the powder to the extruder at a rate of 50 gm/min. The extruder was operated at a speed of 200-300 rpm, at a temperature of 160° C. The residence time in the extruder was 2 to 3 minutes. The melt was extruded through a 2.5-mm die, and then congealed by passing over a chilled belt at a temperature of 15 to 25° C., producing a 3-mm diameter, cylindrical, semi-translucent, hard extrudate. The extrudate was then milled using a Fitz mill in two stages, producing a final product that had a mean particle size of 210 μm. Analysis of the HME by PXRD showed that more than 95% of the HME was non-crystalline.

An in vitro dissolution test was used to determine the dissolution performance of the HME, using the procedures described for the HPMCAS SDD. The concentrations of drug obtained in these samples were used to determine the maximum dissolved concentration of drug and the area under the concentration-versus-time curve during the initial 90 minutes (MDC₉₀ and DAUC₉₀, respectively). The results for the HME and the control are shown in Table 1.

TABLE 1

| | MDC₉₀ | DAUC₉₀ |
|---|---|---|
| Formulation | (μgA/mL) | (min*μgA/mL) |
| HME | 310 | 21,300 |
| Crystalline celecoxib | 60 | 4100 |

The results show that the HME consisting of 34.4 wt % celecoxib, 55.6 wt % HPMCAS-LG, and 10 wt % crospovidone provided concentration-enhancement relative to the crystalline celecoxib alone. The HME provided an MDC₉₀ that was 5.2-fold that provided by crystalline drug alone, and a DAUC₉₀ that was 5.2-fold that provided by crystalline drug alone. Thus, the HME is a solubility-improved form of celecoxib.

### Solubility-Improved Form 4

Nanoparticles (NPs) containing celecoxib were prepared as follows. First, an organic solution was made containing 8.62 wt % celecoxib, 8.62 wt % ethylcellulose ETHOCEL® Viscosity 4, Dow Chemical Co., Midland, Mich.), and 82.76 wt % ethyl acetate. Next, an aqueous solution was made containing 2.04 wt % sodium caseinate and 97.96 wt % water. The organic solution was then poured into the aqueous solution in a 30-L stainless-steel jacketed tank, and homogenized using a Bematek Systems (Salem, Mass.) in-line rotor-stator mixer at 3600 rpm for 20 minutes. This mixture was then homogenized to form an emulsion using an Avestin C55 homogenizer (Ottawa, Ontario), with 20 passes at a pressure of 12,500 psi for 280 minutes. Solvent was removed from the emulsion by heating to 40° C. and drawing a vacuum (with a pressure of 250 mbar) in a mixing tank while stirring for 30 minutes, forming an aqueous suspension of nanoparticles.

The aqueous suspension was spray-dried using a spray dryer to form a solid composition. To form the solid composition, the suspension was pumped using a high-pressure pump to a spray drier (a Niro type XP Portable Spray-Dryer with a Liquid-Feed Process Vessel (“PSD-1”)), equipped with a Schlick #1.0 pressure nozzle (available from Dusen Schlick GmbH of Untersiemau, Germany). The PSD-1 was equipped with a 9-inch chamber extension to increase the vertical length of the dryer. A high-pressure pump was used to deliver liquid to the nozzle. The suspension was pumped to the spray drier at about 24 g/min at a pressure of 300 psig. Drying gas (e.g., nitrogen) at a flow rate of 1850 g/min was delivered at an inlet temperature of 100° C., and the evaporated solvent and drying gas exited the spray drier at a temperature of 50° C. The resulting solid composition was collected in a cyclone, with a mass ratio of 37.5:37.5:25 celecoxib:ethylcellulose: sodium caseinate.

A sample of the solid composition was added to filtered deionized water at a concentration of about 20 mg/mL, and vortexed for 30 seconds. The particle size of the nanoparticles in the aqueous suspension was determined using dynamic light scattering (DLS) as follows. First, the aqueous suspension was filtered using a 1 μm glass membrane filter (Anotop filter, Whatman), and poured into a cuvette. Light-scattering was measured using a Brookhaven Instruments (Holtsville, N.Y.) BI-200SM particle size analyzer with a BI-9000AT correlator. The sums of exponentials from the autocorrelation functions are analyzed to extract size distributions from the samples, and the size is reported as the cumulant value. The average was 135 nm, with a polydispersity of 0.17.

The NP composition was evaluated in vitro using a membrane permeation test as follows. An Accurel® PP 1 E microporous polypropylene membrane was obtained from Membrana GmbH (Wuppertal, Germany). The membrane was washed in isopropyl alcohol and rinsed in methanol in a sonicating bath for one minute at ambient temperature, and then allowed to air dry at ambient temperature. The feed side of the membrane was then plasma-treated to render it hydrophilic by placing a sample of the membrane in a plasma chamber. The atmosphere of the plasma chamber was saturated with water vapor at a pressure of 550 mTorr. A plasma was then generated using radio frequency (RF) power inductively coupled into the chamber via annular electrodes at a power setting of 50 Watts for 45 seconds. The contact angle of a drop of water placed on the surface of the plasma-treated membrane was about 40°. The contact angle of a drop of water placed on the permeate side of the same membrane was greater than about 110°.

A permeate reservoir was formed by capping the open end of a glass tube having an inside diameter of about 2.54 cm by gluing a sample of the plasma-treated membrane to the tube using an epoxy-based glue (LOCTITE® E-30CL HYSOL® from Henkel Loctite Corp, Rocky Hill, Conn.). The membrane was oriented so that its feed side was on the outside of the permeate reservoir and its permeate side was on the inside of the reservoir. The effective membrane area of the membrane capping the permeate reservoir was about 4.9 cm². The permeate reservoir was placed into a glass feed reservoir. The feed reservoir was equipped with a magnetic stir bar and the reservoir was placed on a stir plate and the stir rate was set to 100 rpm during the test. The apparatus was placed into a chamber maintained at 37° C. for the duration of the test.

To form the feed solution, a 13.33 mg sample of the solid NP composition was weighed into the feed reservoir. Five mL of the MFD solution was added to the feed reservoir. The concentration of celecoxib in the feed solution would have been 1000 μg/mL had all of the drug dissolved. The feed solution was mixed using a vortex mixer for one minute. Before the membrane contacted the feed solution, 5 mL of 20 wt % decanol in decane was placed into the permeate reservoir. Time zero in the test was when the membrane was placed in contact with the feed solution. A 50 μl aliquot of the permeate solution was collected at the times indicated. Samples were then diluted in 250 μL IPA and analyzed using HPLC. HPLC analysis was performed using a Zorbax SB C₈ column. The mobile phase consisted of 55% acetonitrile/45% 5 mM triethanolamine, adjusted to pH 7.0. UV absorbance was measured at 254 nm.

As a control, the same test was performed with crystalline celecoxib alone, and 5 mg of the drug was added so that the drug's concentration would have been 1000 μg/ml had all of the drug dissolved.

The maximum flux of drug across the membrane (in units of μg/cm²-min) was determined by performing a least-squares fit to the concentration versus time data from 0 to 60 minutes to obtain the slope, multiplying the slope by the permeate volume (5 mL), and dividing by the membrane area (4.9 cm²). The results of this analysis are summarized in Table 3, and show that the NP formulation provided a maximum flux of celecoxib through the membrane that was 6.6-fold that provided by crystalline drug alone. Thus, the NP formulation is a solubility-improved form of celecoxib.

TABLE 3

| Formulation | Maximum Drug Flux (μg/cm²-min) |
|---|---|
| NP Formulation | 6.6 |
| Crystalline celecoxib | 1.0 |

### Immediate Release (IR) Granulations

### Immediate Release Granulation 1 (IRG-1)

IRG-1 was made using celecoxib solubility-improved form 1 (the HPMCAS SDD) according to the composition shown in Table 4. The intragranular components were weighed out for a final batch size of 3750 g. The Celecoxib HPMCAS-LG SDD, microcrystalline cellulose (Avicel® PH102, FMC Corporation, Philadelphia, Pa.), lactose (Fast Flo 316® Spray Dried, Foremost Farms, Baraboo, Wis.) and the crospovidone (Polyplasdone® XL, International Specialty Products, Wayne, N.J.) were added to a 16 quart twin-shell blender (Patterson-Kelley Co., East Stroudsburg, Pa.) and blended for 15 minutes. The intragranular magnesium stearate (Mallinckrodt Inc, St. Louis, Mo.) was added to the blender and blended an additional 5 minutes. The blend was transferred to a Gerteis® Mini-pactor®(Gerteis Maschinen+Processengineering AG, Jona, Switzerland) equipped with knurled rolls, polished side rims, a pocket rotor and a 0.8 mm screen. The roller compactor was run at the following settings: roll pressure 7 kN/cm, roll gap 2 mm, roll speed 2 rpm, a feed auger to tamp auger ratio at 2.5:1, rotor speed 80 rpm, and rotor rotation at 180° counter clockwise/720° clockwise. The weight of milled granulation was determined and the appropriate amount of extragranular magnesium stearate was weighed out. The milled granulation and the magnesium stearate were added to the 16 quart twin-shell blender and blended 5 minutes.

TABLE 4

| Component | Mass (wt %) |
|---|---|
| Intragranular | |
| Celecoxib HPMCAS-LG SDD (50% A) | 60.00 |
| Microcrystalline Cellulose (Avicel PH102) | 23.00 |
| Lactose (Fast Flo 316 Spray Dried) | 11.50 |
| Crospovidone (Polyplasdone XL) | 5.00 |
| Magnesium Stearate | 0.25 |
| Extragranular | |
| Magnesium Stearate | 0.25 |
| Total | 100.00 |

### IR Granulation 2 (IRG-2)

IRG-2 was made using celecoxib solubility-improved form 1 (the HPMCAS SDD) according to the composition shown in Table 5. The intragranular components were weighed out for a final batch size target of 1500 grams. The Celecoxib HPMCAS SDD, microcrystalline cellulose (Avicel PH102) and croscarmellose sodium (Ac-Di-Sol®, FMC Corporation, Philadelphia, Pa.) were added to an 8 quart twin-shell blender (Patterson-Kelley Co.) and blended for 15 minutes. The intragranular magnesium stearate was added to the blender and blended an additional 5 minutes. The blend was discharged and transferred to a Gerteis Minipactor and processed as in IRG-1. From the milled granulation, a 125 g final blend was prepared. Accordingly, a 101.98 g sample of the granulation, 18.96 g of microcrystalline cellulose (Avicel PH200) and 3.75 g croscarmellose sodium (Ac-Di-Sol) were placed in a 900 cc bottle. The contents were blended in a Turbula® T2F mixer (Willy A. Bachofen AG Maschinenfabrik, Basel, Switzerland) mixer for 10 minutes. Finally, 0.313 g of magnesium stearate was added to the bottle and the contents were blended an additional 3 minutes in the Turbula mixer.

TABLE 5

| Components | Mass (wt %) |
|---|---|
| Intragranular | |
| Celecoxib HPMCAS SDD (50% A) | 40.00 |
| Microcrystalline Cellulose (Avicel PH102) | 36.33 |
| Croscarmellose sodium (Ac-Di-Sol) | 5.00 |
| Magnesium Stearate | 0.25 |
| Extragranular components | |
| Microcrystalline Cellulose (Avicel PH200) | 15.17 |
| Croscarmellose sodium (Ac-Di-Sol) | 3.00 |
| Magnesium Stearate | 0.25 |
| Total | 100 |

### IR Granulation 3 (IRG-3)

IRG-3 was made using celecoxib solubility-improved form 2 (the HPMC SDD) according to the composition shown in Table 6, using the procedures outlined for IRG-1 with the following exception: the roll pressure was set at 8 kN/cm.

TABLE 6

| Components | Mass (wt %) |
|---|---|
| Intragranular | |
| Celecoxib HPMC SDD (50% A) | 40.00 |
| Microcrystalline Cellulose (Avicel PH102) | 36.33 |
| Crospovidone (Polyplasdone XL) | 5.00 |
| Magnesium Stearate | 0.25 |
| Extragranular components | |
| Microcrystalline Cellulose (Avicel PH200) | 15.17 |
| Crospovidone (Polyplasdone XL) | 3.00 |
| Magnesium Stearate | 0.25 |
| Total | 100 |

### IR Granulation 4 (IRG-4)

IRG-4 was made using celecoxib solubility-improved form 2 (the HPMC SDD) according to the composition shown in Table 7, using the procedures outlined for IRG-2 with the following exception: the roll pressure was set at 8 kN/cm, and the rotor speed was set at 50 rpm.

TABLE 7

| Components | Mass (wt %) |
|---|---|
| Intragranular | |
| Celecoxib HPMC SDD (50% A) | 40.00 |
| Microcrystalline Cellulose (Avicel PH102) | 36.03 |
| Crosscarmellose sodium (Ac-Di-Sol) | 5.00 |
| Magnesium Stearate | 0.25 |
| FD&C Blue #2Lake | 0.30 |
| Extragranular components | |
| Lactose (Fast Flo 316 Spray Dried) | 15.17 |
| Crosscarmellose sodium (Ac-Di-Sol) | 3.00 |
| Magnesium Stearate | 0.25 |
| Total | 100 |

### Sustained Release Granulations

### Sustained Release Granulation 1 (SRG-1)

SRG-1 was made using celecoxib solubility-improved form 1 (the HPMCAS SDD) according to the composition shown in Table 8. The intragranular components were weighed out for a final batch size of 9200 g. The Celecoxib HPMCAS-LG SDD, hypromellose (Methocel™ K100LV Premium CR, Dow Chemical Co., Midland, Mich.) and lactose (Fast Flo 316 Spray Dried) were added to a 1 cubic foot twin-shell blender (Patterson-Kelley Co.) and blended for 15 minutes. The intragranular magnesium stearate was added to the blender and blended an additional 5 minutes. The blend was transferred to a Gerteis Minipactor equipped with knurled rolls, polished side rims, a pocket rotor and a 0.8 mm screen. The roller compactor was run at the following settings: roll pressure 7 kN/cm, roll gap 2 mm, roll speed 2 rpm, a feed auger to tamp auger ratio of 2.5:1, rotor speed 80 rpm, and rotor rotation of 180° counter clockwise/720° clockwise. The weight of milled granulation was determined and the appropriate amount of extragranular magnesium stearate was weighed out. The milled granulation and the magnesium stearate were added to the 1 cubic foot blender and blended 5 minutes.

TABLE 8

| Components | Mass (wt %) |
|---|---|
| Intragranular | |
| Celecoxib HPMCAS SDD (50% A) | 60.00 |
| Hypromellose (Methocel K100LV Premium CR) | 30.00 |
| Lactose (Fast Flo 316 Spray Dried) | 9.50 |
| Magnesium Stearate | 0.25 |
| Extragranular components | |
| Magnesium Stearate | 0.25 |
| Total | 100 |

### Sustained Release Granulation 2 (SRG-2)

SRG-2 was made using celecoxib solubility-improved form 2 (the HPMC SDD) according to the composition shown in Table 9. The intragranular components were weighed out for a final batch size of 1500 g. The Celecoxib HPMC SDD, hypromellose (Methocel™ K4M, Dow Chemical Co.), and the Carbopol® (Noveon Inc., Cleveland, Ohio) were added to an 8 quart twin-shell blender and blended for 15 minutes. The intragranular magnesium stearate was added to the blender and the batch was blended an additional 5 minutes. The blend was transferred to a Gerteis Minipactor equipped with knurled rolls, polished side rims, a pocket rotor and a 0.8 mm screen. The roller compactor was run at the following settings: roll pressure 6.5 kN/cm, roll gap 2 mm, roll speed 2 rpm, a feed auger to tamp auger ratio at 1.5:1, rotor speed 90 rpm, and rotor rotation at 120° counter clockwise/120° clockwise. The weight of milled granulation was determined and the appropriate amount of extragranular lactose (Fast Flo 316 Spray Dried) was added to the granulation and blended in the twin-shell blender for 15 minutes. The extragranular magnesium stearate was weighed out and added to the blender and the mixture was blended for 5 minutes.

TABLE 9

| | Mass (wt %) | | |
|---|---|---|---|
| Component | SRG-2 | SRG-3 | SRG-4 |
| Intergranular | | | |
| Celecoxib HPMC SDD (50% A) | 40.00 | 40.00 | 40.00 |
| Hypromellose (Methocel K100LV Prem) | 30.00 | 0.00 | 0.00 |
| Hypromellose (Methocel K4M) | 0.00 | 40.00 | 30.00 |
| Carbopol 71G | 0.00 | 0.00 | 1.00 |
| Magnesium Stearate | 0.25 | 0.25 | 0.25 |
| Extragranular | | | |
| Lactose, Fast Flo 316 Spray Dried | 29.50 | 19.50 | 28.50 |
| Magnesium Stearate | 0.25 | 0.25 | 0.25 |
| Total | 100.00 | 100.00 | 100.00 |

### Sustained Release Granulations 3-4 (SRG-3 and SRG-4)

SRG-3 and SRG-4 were prepared using celecoxib solubility-improved form 2 (the HPMC SDD) according to the compositions shown in Table 9 using the procedures outlined for SRG-2.

### Sustained Release Granulation 5 (SRG-5)

SRG-5 was formed using celecoxib solubility-improved form 3 (the HME) as follows. A portion of the HME was blended with crospovidone (as a disintegrant), hydroxypropyl cellulose (HPC, as a binder), Pluronic® F-108 (as a surfactant, BASF Corporation, Florham Park, N.J.), and magnesium stearate (as lubricant) to obtain the blend composition shown in Table 10.

TABLE 10

| Component | | Mass (mg/tablet) | Mass (wt %) |
|---|---|---|---|
| HME | Celecoxib | 160.00 | 27.7 |
| | HPMCAS | 258.8 | 44.8 |
| | Crospovidone | 46.8 | 8.0 |

| | | |
|---|---|---|
| Crospovidone | 28.9 | 5.0 |
| Pluronic F-108 | 23.1 | 4.0 |
| HPC | 57.8 | 10.0 |
| Mg stearate | 2.9 | 0.5 |
| Total | 578.3 | 100.0 |

### Exemplary Dosage Forms

### Dosage Form 1 (DF-1)

DF-1 was made on a Korsch XL400 bilayer press (Korsch America Inc, South Easton, Mass.) equipped with 35 stations of 0.3295×0.6450 inch modified oval tooling running at a turret speed of 30 rpm. The SR portion was filled first with a target weight of 417 mg of SRG-1 and lightly tamped to ensure a distinct interface between the layers. After tamping the hardness of the SR portion was less than 1 kiloponds (kP). The IR portion was filled second with a target weight of 167 mg of IRG-1. The average tablet weight was 584 mg and had a hardness of 12-13 kP. The resulting dosage form had 50 mgA celecoxib in the IR portion and 125 mgA celecoxib in the SR portion.

DF-1 was tested in vitro in a USP Apparatus II (Vankel VK700 dissolution bath, Varian, Inc., Cary, N.C.) with baskets at 100 rpm in 1000 mL of 50 mM sodium phosphate adjusted to pH 6.8 containing 2% w/v sodium dodecyl sulfate (SDS). Celecoxib dissolution was determined by in situ fiber optic probes (Rainbow Dynamic Dissolution Monitor™, Delphian Technology Inc., Woburn, Mass.) set at an analytical wavelength of 254 nm and a baseline correction wavelength of 320 nm. Dissolution results are given in Table 11. At 30 minutes 28% of celecoxib was dissolved in the media. At 7 hours approximately 80% of the celecoxib was dissolved.

TABLE 11

| Time (hours) | Drug Dissolved (wt %) |
|---|---|
| 0 | 0 |
| 0.25 | 23 |
| 0.5 | 28 |
| 0.75 | 32 |
| 1 | 36 |
| 2 | 46 |
| 3 | 53 |
| 5 | 65 |
| 7 | 77 |
| 9 | 88 |
| 12 | 98 |

### Dosage Form 2 (DF-2)

DF-2 was made on a single station Manesty F-press (Manesty, Merseyside, United Kingdom) equipped with 0.3395×0.6790 inch modified oval tooling. The SR portion consisted of 500 mg of SRG-4. The press was rotated by hand to lightly depress the SR portion and provide the space required for the IR granulation. The IR portion consisted of 200 mg of IRG-2. The tablet was then fully compressed. The final tablet thickness was approximately 6.6 mm. The tablets contained 100 mgA of Celecoxib in the SR portion and 40 mgA of Celecoxib in the IR portion.

DF-2 was tested using the procedures outlined for DF-1. The results are presented in Table 12.

TABLE 12

| Time (hours) | Drug Dissolved (wt %) |
|---|---|
| 0 | 0 |
| 0.25 | 13 |
| 0.5 | 18 |
| 0.75 | 22 |
| 1 | 26 |
| 2 | 36 |
| 3 | 40 |
| 5 | 50 |
| 7 | 56 |
| 9 | 62 |
| 12 | 71 |

### Dosage Form 3 (DF-3)

DF-3 was made using the procedures used to form DF-2 with the following exceptions. The SR portion consisted of 333.3 mg SRG-1, while the IR portion consisted of 200 mg of IRG-3. DF-3 contained 100 mgA celecoxib in the SR portion and 40 mgA celecoxib in the IR portion. The final tablet thickness was approximately 5.2 mm.

DF-3 was tested using the procedures outlined for DF-1. The results are presented in Table 13.

TABLE 13

| Time (hours) | Drug Dissolved (wt %) |
|---|---|
| 0 | 0 |
| 0.25 | 27 |
| 0.5 | 31 |
| 0.75 | 35 |
| 1 | 39 |
| 2 | 52 |
| 3 | 65 |
| 5 | 90 |
| 7 | 99 |
| 9 | 98 |
| 12 | 97 |

### Dosage Forms 4-6 (DF-4, DF-5, DF-6)

Dosage forms 4-6 were made using the procedures used to form DF-2 with the following exceptions. For DF-4, the SR portion consisted of 333.3 mg SRG-2, while the IR portion consisted of 200 mg of IRG-4. For DF-5, the SR portion consisted of 333.3 mg SRG-3, while the IR portion consisted of 200 mg of IRG-4. For DF-6, the SR portion consisted of 333.3 mg SRG-4, while the IR portion consisted of 200 mg of IRG-4.

These dosage forms all contained 100 mgA celecoxib in the SR portion and 40 mgA celecoxib in the IR portion.

The dosage forms were tested using the procedures outlined for DF-1. The results are presented in Table 14.

TABLE 14

| | Drug Dissolved (wt %) | | |
|---|---|---|---|
| Time (hours) | DF-4 | DF-5 | DF-6 |
| 0 | 0 | 0.0 | 0 |
| 0.5 | 26 | 22 | 19 |
| 1 | 37 | 31 | 26 |
| 2 | 53 | 41 | 35 |
| 3 | 66 | 48 | 40 |
| 5 | 90 | 60 | 49 |
| 7 | 100 | 71 | 56 |
| 9 | — | 81 | 64 |
| 12 | — | 92 | 76 |
| 15 | — | 99 | 86 |
| 20 | — | — | 100 |

### Dosage Form 7 (DF-7)

DF-7 was made using the procedures used to form DF-2 with the following exceptions. The SR portion consisted of 333.3 mg SRG-1, while the IR portion consisted of 200 mg of IRG-4. DF-7 contained 100 mgA celecoxib in the SR portion and 40 mgA celecoxib in the IR layer.

### Dosage Form 8 (DF-8)

DF-8 was made using the procedures used to form DF-2 with the following exceptions. The SR portion consisted of 333.3 mg SRG-5, while the IR portion consisted of 200 mg of IRG-4. DF-8 contained 100 mgA celecoxib in the SR portion and 40 mgA celecoxib in the IR portion.

### Model Discussion

A mathematical model (GastroPlus™) was developed to explore the input rates for celecoxib dosage forms that would meet certain in vivo release targets. GastroPlus™ is a computer program that simulates absorption and pharmacokinetics for orally dosed drugs. The underlying model is the Advanced Compartmental Absorption and Transit (ACAT) model—an extension of work originally done by Gordon Amidon and Lawrence Yu. See L. X. Yu, “An Integrated Model for Determining Causes of Poor Oral Drug Absorption,” Pharm. Res., 16:1883-7 (1999) and B. Agoram, W. S. Woltosz, and M. B. Bolger, “Predicting the impact of physiological and biochemical processes on oral drug bioavailability,” Advanced Drug Delivery Reviews, 50:S41-S67 (2001).

GastroPlus™ was used to simulate the absorption and pharmacokinetics of the reference and test formulations. The program has three input pages: compound, physiology, and pharmacokinetics. In the compound page, basic data of the drug's physical and chemical properties such as bulk density (1.2 g/mL), solubility (0.0116 mg/mL at pH 7), pKa (11.1), and particle size distribution are entered. The human permeability (P_{eff}) of celecoxib was estimated to be 1.1262×10⁻⁴ cm/s based on clinical data. The diffusion coefficient of celecoxib was estimated by GastroPlus™ to be 0.6752×10⁻⁵ cm²/sec.

The in vitro dissolution profiles of celecoxib formulations were used as input functions to simulate the absorption and pharmacokinetics of the reference (Celebrex commercial capsule) and test formulations. The drug release profiles were used by the software to calculate the drug concentration in each compartment. The estimated human permeability data were computed using a modification of the human fasted log D absorption model to account for permeability. The model then calculated the fraction of the dose absorbed based on the ACAT model using drug concentration, permeability, surface area, and transit time in each compartment. Pharmacokinetic parameters, e.g. volume of distribution, clearance, and micro-constants were added to the software in the pharmacokinetic page, which enabled the software to calculate plasma concentration—time curves.

In the physiology page, a user-supplied physiology file was provided to the program as an input file, consisting of the following transit times for each compartment (hr): 0.1, 0.25, 0.5, 0.5, 1.0, 1.0, 1.0, 8.0, and 24.0 hr.

The suitability of the model to predict in vivo performance was evaluated by comparing modeling results to in vivo results using commercial capsules containing 200 mgA celecoxib. In this test, 12 human subjects fasted from at least 10 hours predose until 4 hours postdose. Water was restricted for 1 hour before and after dosing, except for the volume (240 mL or 8 fluid ounces) administered with the dose. Each subject was administered 400 mgA celecoxib as 2×200 mgA commercial celecoxib capsules. Blood samples for analysis of plasma celecoxib concentrations were collected predose, and at 0.25, 0.5, 0.75, 1, 1.5, 2, 3, 4, 6, 8, 12, 24, and 48 hours following dosing.

Plasma samples were analyzed for celecoxib using a validated, sensitive and specific high-performance liquid chromatography tandem mass spectrometric method (HPLC/MS/MS) method. The plasma specimens were stored at approximately −20° C. until assay and samples were assayed within the 343 days of established stability data generated during validation. Calibration standard responses were linear over the range of 1.00 to 2000 ng/mL using a linear, weighted (1/concentration squared) linear regression. Those samples with concentrations above the upper limits of quantification were adequately diluted into calibration range. The lower limit of quantification (LLOQ) for celecoxib was 1.00 ng/mL. Clinical specimens with plasma celecoxib concentrations below the LLOQ were reported as below lower limit of quantification (1.00 ng/mL).

FIG. 2 shows the mean plasma celecoxib concentration versus time for this test. Also shown are the results of using the model to simulate this dosage form. These results show excellent agreement between the model and the in vivo results.

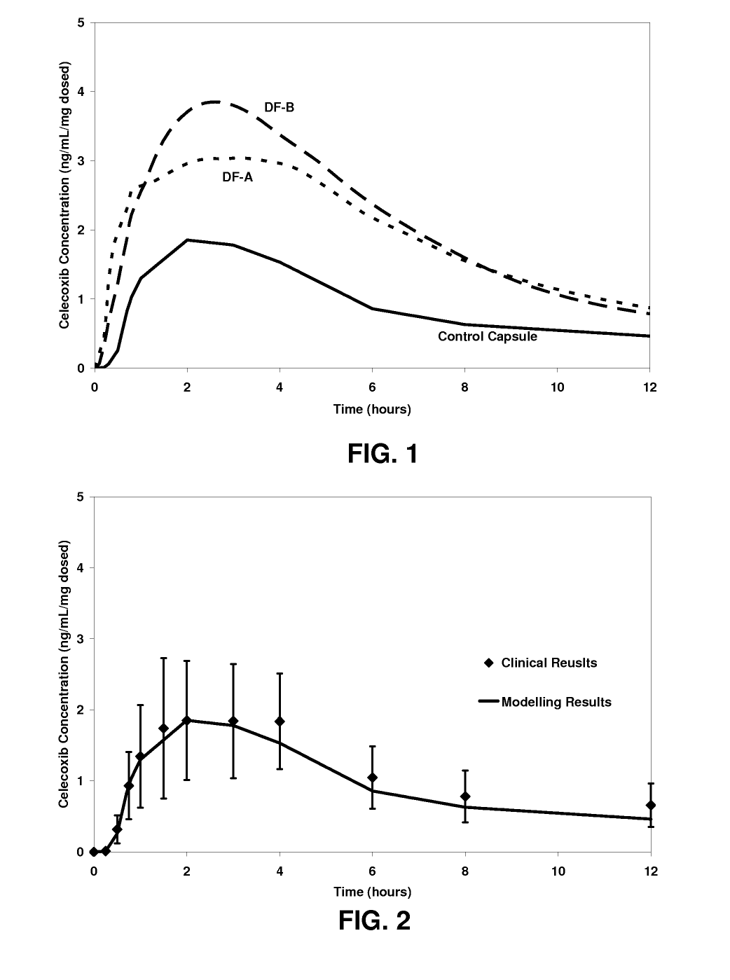

The model was used to evaluate the performance of the dosage forms of the invention. The results of this modeling work are shown in FIG. 1. Here, DF-A is a dosage form similar to dosage forms DF-4, DF-5, and DF-6, while DF-B is similar to dosage form DF-1. These data show that the dosage forms of the invention will be effective.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1-33. (canceled)

34. A pharmaceutical dosage form comprising celecoxib and a pharmaceutically acceptable carrier, comprising:
(a) an immediate release portion;
(b) a sustained release portion comprising celecoxib in a solubility-improved form.

35. The dosage form of claim 34 wherein said immediate release portion comprises celecoxib in a solubility-improved form.

36. The dosage form of claim 35 wherein said solubility-improved form in said immediate release portion is the same as said solubility-improved form in said sustained release portion.

37. The dosage form of claim 34 wherein said solubility-improved form is selected from the group consisting of molecular dispersions, and nanoparticles.

38. The dosage form of claim 34 wherein said solubility-improved form is an amorphous solid dispersion of celecoxib in a polymer.

39. The dosage form of claim 38 wherein said polymer is hydroxypropyl methylcellulose acetate succinate (HPMCAS).

40. The dosage form of claim 38 wherein said polymer is hydroxypropyl methylcellulose (HPMC).

41. The dosage form of claim 34 wherein said sustained release portion comprises a matrix.

42. The dosage form of claim 41 wherein said matrix is selected from the group consisting of chitin, chitosan, dextran, pullulan, gum agar, gum arabic, gum karaya, locust bean gum, gum tragacanth, carrageenans, gum ghatti, guar gum, xanthan gum, scleroglucan, dextrin, maltodextrin, pectin, lecithin, ammonium alginate, sodium, potassium or calcium alginate, propylene glycol alginate, gelatin, collagen, ethyl cellulose, methylethyl cellulose, carboxymethyl cellulose, carboxymethyl ethylcellulose, hydroxyethyl cellulose, hydroxypropyl cellulose, cellulose acetate phthalate, cellulose acetate trimellitate, hydroxypropyl methyl cellulose, hydroxypropyl methyl cellulose phthalate, hydroxypropyl methyl cellulose acetate succinate, hydroxypropyl methyl cellulose acetate trimellitate, ethylhydroxy ethylcellulose, and mixtures thereof.

43. The dosage form of claim 34 comprising from 20 to 300 mg celecoxib.

44. The dosage form of claim 34 wherein said immediate release portion comprises from 20 to 80 mg celecoxib.

45. The dosage form of claim 34 wherein said sustained release portion comprises from 50 to 200 mg celecoxib.

46. A pharmaceutical dosage form comprising celecoxib, said dosage form comprising
(a) 25 to 60 wt % of an immediate release portion comprising
(i) 30 to 80 wt % of a molecular dispersion of celecoxib and a polymer selected from the group consisting of HPMC and HPMCAS;
(ii) 1 to 15 wt % disintegrant;
(iii) 20 to 60 wt % diluent; and
(iv) 0.05 to 2 wt % lubricant; and
(b) 40 to 75 wt % of a sustained release portion comprising
(i) 30 to 80 wt % of a molecular dispersion of celecoxib and a polymer selected from the group consisting of HPMC and HPMCAS;
(ii) 10 to 50 wt % matrix material;
(iii) 2 to 40 wt % diluent; and
(iv) 0.05 to 2 wt % lubricant.

47. The dosage form of claim 46 wherein said molecular dispersion of said immediate release portion comprises from 40 to 60 wt % celecoxib in HPMC.

48. The dosage form of claim 47 wherein said molecular dispersion of said sustained release portion comprises from 40 to 60 wt % celecoxib in HPMC.

49. The dosage form of claim 46 wherein said immediate release portion comprises
(1) 35 to 45 wt % of said molecular dispersion of celecoxib and said polymer;
(2) 30 to 40 wt % microcrystalline cellulose;
(3) 2 to 7 wt % croscarmellose sodium; and
(4) 0.05 to 1 wt % magnesium stearate.

50. The dosage form of claim 46 wherein said sustained release portion comprises
(1) 35 to 45 wt % of said molecular dispersion of celecoxib and said polymer;
(2) 25 to 45 wt % hydroxypropyl methyl cellulose;
(3) 15 to 35 wt % lactose; and
(4) 0.05 to 1 wt % magnesium stearate
